(19) United States
(12) Patent Application Publication
(10) Pub. No.: US 2008/0108963 A1
(43) Pub. Date: May 8, 2008

(54) Absorbent Article With Handles Of Slow Recovery Material

(75) Inventors: Gregory Ashton, Cincinnati, OH (US); Massaharu Nishikawa, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/934,115

(22) Filed: Nov. 2, 2007

Related U.S. Application Data
(60) Provisional application No. 60/856,131, filed on Nov. 2, 2006.

(51) Int. Cl. A61F 13/49; A61F 13/84

(52) U.S. Cl. 604/385.01; 604/378

(57) ABSTRACT

An absorbent article includes a chassis with a backsheet, a topsheet joined to the backsheet, and an absorbent core disposed between the backsheet and the topsheet. The article also includes at least one handle joined to the chassis. The handle is of a material that is capable of transmitting at least 30-50% of the force applied and that recovers to substantially or nearly substantially its initial shape after forces are applied to the handle are removed. The material may be a slow recovery elastomer with a normalized unload force of greater than about 0.04 N/mm² at 37C after a 5 minute hold time at 60% strain and a post-elongation strain of at least about 30% after 15 seconds of recovery at 22C. A method of use of the article is also included.

### CROSS REFERENCE TO RELATED APPLICATION

This application claims the benefit of U.S. Provisional Application No. 60/856,131, filed Nov. 2, 2006, the substance of which is incorporated herein by reference.

### FIELD OF THE INVENTION

The present disclosure is directed to an absorbent article, such as a training pant or the like, that has handles attached thereto, and in particular to an absorbent article with handles formed, at least in part, of a slow recovery material.

### BACKGROUND OF THE INVENTION

Absorbent articles, such as diapers and training pants, are well known in the art. These articles typically have an absorbent assembly held or positioned in proximity to the body of a wearer during use in order to capture and absorb bodily exudates discharged from the wearer. Typical absorbent articles include a topsheet facing the wearer, which permits fluid exudates to pass through to an absorbent core, and a backsheet, which prevents the exudates from escaping from the absorbent article.

Typically, a child will begin wearing a diaper soon after birth. At some point, the caregiver will look to transition the child away from wearing diapers. This transition is typically referred to as toilet training, and typically involves the child learning to recognize when he or she needs to use the toilet because the discharge of exudates is imminent.

During the time the child is toilet training, the child may still wear an absorbent article, in the form of a training pant or the like. In one sense, the training pant provides a function similar to the diaper, in that it limits the escape of exudates from the absorbent article. In another sense, relative to the wearer, the focus may be slightly different. While the focus of the diaper may be to limit exposure of the infant's skin to exudates, the focus of the training pant may be to create or to simulate a feeling of wetness or coolness for the wearer, so that the wearer may begin to associate certain urges and feelings with the discharge of exudates. Hopefully, the association of the urges and the discharges will permit the wearer to predict when he or she needs to use the toilet, and thereby make the transition from the absorbent article.

Toilet training usually does not occur immediately, and may take several days or weeks to complete. Even after the child is “toilet trained” relative to daytime activity, it may still be necessary for the child to wear a pant during the nighttime. That is, certain children, for physiological or psychological reasons, may be capable of predicting discharge during the daytime, but may remain unable to do so at night. For these children, the time during which they wear an absorbent article may thus be extended relative to other children.

It is also typically the case that the age at which children are toilet trained in “developed” countries has increased steadily over the past several decades. Now the age at which children are toilet trained is in the range of about 24-48 months. Nighttime use of pants may extend the age even further. As a consequence, the toddler that is toilet training or wearing pants at nighttime may be sufficiently mature to assist the caregiver in putting on and taking off their own training pants or nighttime pants, much in the same way the child may be developing the skills to dress and undress him or herself.

While the child may be mature enough to want to take responsibility for putting on and taking off the training or the nighttime pants, the child may not have developed sufficient skills to perform this activity independently. Not all children develop motor skills at the same rate, and certain skills more easily learned than others. Furthermore, it may be the case that the caregiver may need to assist the toddler in putting on the pants, which may require the caregiver to bend or stoop to the level of the child. All of these factors may cause discomfort, discouragement, or frustration with the process on the part of the toddler, the caregiver, or both.

It has been proposed to add a handle or strap to the pant to assist the child or the caregiver in putting on the pant. These handles or straps may be used by the child or the caregiver to limit the amount of stooping or bending necessary when the pant is placed on the floor to let the child to put his or her feet through the leg openings in the pant. Moreover, it may be easier for the child (or the caregiver) to grip the handle or strap, than it was for the child (or caregiver) to grip the sides of the pant.

Unfortunately, these handles or straps may have undesired cosmetic or functional effects. For instance, while the handles or straps may be folded against the sides of the pant prior to use, once the handles or straps are used, the handles or straps may remain loose from the sides of the pant. As a consequence, the handle or strap must be tucked into the waist of the pant, which may be uncomfortable for child, or the handle or strap may hang loosely to the side of the pant, which may provide an unkempt appearance. Alternatively, if the handle or strap is made of stretchy elastic material, so the handle or strap will return to a position close to the body after use, it may be difficult to transfer sufficient forces to the pant while using the handles to move the pant up into its operative position on the wearer.

Accordingly, it would be desirable to provide a system or an article that facilitates efforts of the user to put on an absorbent article. It would also be desirable to provide a system or an article that facilitates the efforts of the user while overcoming one or more of the drawbacks of conventional handle or strap technology.

### SUMMARY OF THE INVENTION

In a first aspect, an absorbent article includes a chassis with a backsheet, a topsheet joined to the backsheet, and an absorbent core disposed between the backsheet and the topsheet. The article also includes at least one handle joined to the chassis. The handle is of a slow recovery elastomer, the slow recovery elastomer exhibiting a normalized unload force of greater than about 0.04 N/mm2 at 37C after a 5 minute hold time at 60% strain and a post-elongation strain of at least about 30% after 15 seconds of recovery at 22C.

In a second aspect, an absorbent article includes a chassis with a backsheet, a topsheet joined to the backsheet, and an absorbent core disposed between the backsheet and the topsheet. The article also includes at least one handle joined to the chassis. The handle is of a material that is capable of transmitting at least 30-50% of the force applied and that recovers to substantially or nearly substantially its initial shape after forces are applied to the handle are removed.

In a third aspect, a method of using an absorbent article is provided. The absorbent article includes a chassis with a backsheet, a topsheet joined to the backsheet, and an absorbent core disposed between the backsheet and the topsheet, the chassis fastened to define leg openings and a waist opening. The article also includes at least one handle joined to the chassis and shaped to facilitate grasping by one of limited dexterity. The method includes disposing a wearer's legs through the waist opening and the leg openings, grasping the at least one handle, applying a force to the chassis via the at least one handle to move the article upward along the legs toward the wearer's waist, at least 30-50% of the force applied to the at least one handle being transmitted to the chassis, and releasing the at least one handle, the at least one handle subsequently recovering to substantially or nearly substantially its initial shape.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a plan view of an absorbent article according to the present disclosure in a flat, uncontracted state;

FIG. 2 is a side perspective view of the absorbent article of FIG. 1 illustrating a first embodiment of a handle according to the present disclosure;

FIG. 3a is a side perspective view of the absorbent article of FIG. 2 illustrating the handle in a first position or a first state;

FIG. 3b is a side perspective view of the absorbent article of FIG. 2 illustrating the handle in a second position or a second state;

FIG. 3c is a side perspective view of the absorbent article of FIG. 2 illustrating the handle in a third position or a third state;

FIG. 3d is a side perspective view of the absorbent article of FIG. 2 illustrating the handle in a fourth position or a fourth state;

FIG. 3e is a side perspective view of the absorbent article of FIG. 2 illustrating the handle in a fifth position or a fifth state;

FIG. 4 is a side perspective view of the absorbent article of FIG. 1 illustrating a second embodiment of a handle according to the present disclosure;

FIG. 5a is a side perspective view of the absorbent article of FIG. 1 illustrating a third embodiment of a handle according to the present disclosure;

FIG. 5b is a side perspective view of the absorbent article of FIG. 1 illustrating a fourth embodiment of a handle according to the present disclosure;

FIG. 6a is a side perspective view of the absorbent article of FIG. 1 illustrating a fifth embodiment of a handle according to the present disclosure;

FIG. 6b is a cross-sectional view of the absorbent article of FIG. 6a taken along line 6b-6b in FIG. 6a;

FIG. 6c is a cross-sectional view illustrating a sixth embodiment of a handle according to the present disclosure;

FIG. 6d is a cross-sectional view illustrating a seventh embodiment of a handle according to the present disclosure;

FIG. 7 is a side perspective view of the absorbent article of FIG. 1 illustrating an eighth embodiment of a handle according to the present disclosure;

FIG. 8a is a side perspective view of the absorbent article of FIG. 1 illustrating a ninth embodiment of a handle according to the present disclosure;

FIG. 8b is a side perspective view of the absorbent article of FIG. 1 illustrating a tenth embodiment of a handle according to the present disclosure;

FIG. 9a is a side perspective view of the absorbent article of FIG. 1 illustrating an eleventh embodiment of a handle according to the present disclosure; and

FIG. 9b is a side perspective view of the absorbent article of FIG. 1 illustrating a twelfth embodiment of a handle according to the present disclosure.

### DETAILED DESCRIPTION OF THE INVENTION

### Definitions

As used herein, the following terms shall have the meaning specified thereafter:

“Absorbent article” refers to devices which absorb and contain body exudates and, more specifically, refers to devices which are placed against or in proximity to the body of the wearer to absorb and contain the various exudates discharged from the body. Exemplary absorbent articles include diapers, training pants, article pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Disposable” (in reference to absorbent articles) means that the absorbent articles are generally not intended to be laundered or otherwise restored or reused as absorbent articles (i.e., they are intended to be discarded after a single use and, preferably, to be recycled, composted or otherwise discarded in an environmentally compatible manner).

“Elastic,” “elastomer,” and “elastomeric” refer to a material which generally is able to extend to a strain of at least 50% without breaking or rupturing, and is able to recover substantially to its original dimensions after the deforming force has been removed.

“Elastomeric material” is a material exhibiting elastic properties. Elastomeric materials may include elastomeric films, scrims, nonwovens, and other sheet-like structures.

“Extendibility” and “extensible” mean that the width or length of the component in a relaxed state can be extended or increased.

“Film” refers to a sheet-like material wherein the length and width of the material far exceed the thickness of the material. Typically, films have a thickness of about 0.5 mm or less.

“Joined” refers to configurations whereby an element is directly secured to another element by affixing the element directly to the other element and to configurations whereby an element is indirectly secured to another element by affixing the element to intermediate member(s) which in turn are affixed to the other element.

“Laminated structure” or “laminate” (unless otherwise noted) means a structure in which one layer, material, component, web, or substrate is adhesively bonded, at least in part, to another layer, material, component, web, or substrate. As stated elsewhere in this application, a layer, material, component, web, or substrate may be folded over and adhesively bonded to itself to form a “laminated structure” or “laminate.”

“Lateral” refers to a direction running from a longitudinal edge to an opposing longitudinal edge of the article and generally at a right angle to the longitudinal direction. Directions within 45 degrees of the lateral direction are considered to be “lateral.”

“Longitudinal” refers to a direction running substantially perpendicular from a waist edge to an opposing waist edge of the article and generally parallel to the maximum linear dimension of the article. Directions within 45 degrees of the longitudinal direction are considered to be “longitudinal.”

“Nonwoven” fabric or web means a web having a structure of individual fibers or threads that are interlaid, but not in a regular or identifiable manner as in a knitted fabric. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note: to convert from osy to gsm, multiply osy by 33.91.)

“Outboard” and “inboard” refer respectively to the location of an element disposed relatively far from or near to the longitudinal centerline of the diaper with respect to a second element. For example, if element A is outboard of element B, then element A is farther from the longitudinal centerline than is element B.

“Pant” refers to disposable absorbent articles which may have waist and leg openings formed prior to application, for example, by the manufacturer. A pant may be donned by inserting a wearer's legs into the leg openings and sliding the pant into position about the wearer's lower torso. Pants are also commonly referred to as “closed diapers”, “prefastened diapers”, “pull-on diapers”, “training pants” and “diaper-pants.”

“Proximal” and “distal” refer respectively to the location of an element relatively near to or far from the longitudinal or lateral centerline of a structure (e.g., the proximal edge of a longitudinally extending element is located nearer to the longitudinal centerline than the distal edge of the same element is located relative to the same longitudinal centerline).

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable.” As is well known in the art, a common method for measuring the permeability to water, urine, or synthetic urine of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

### Absorbent Article and Kit

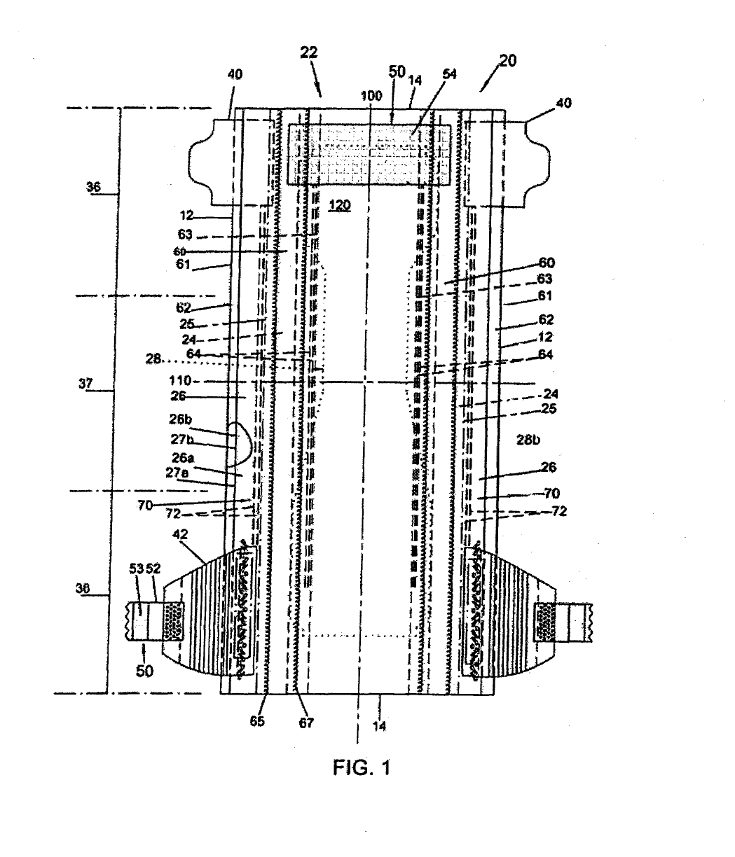

FIG. 1 is a plan view of an exemplary, non-limiting embodiment of an absorbent article 20, which could be a diaper or a pant. As will be explained with reference to FIGS. 2-9, the article 20 will be used in combination with one or more handles 200 to assist the wearer of the article 20 or a caregiver in positioning the article 20 on the wearer. While it may be expected that the article 20 will be in the form of a pant, i.e., with waist and leg openings formed prior to positioning, when the handles 200 are used, this is not to suggest that the article 20 need be preformed as a pant by manufacturer. Some assembly of the article may be required after manufacture but prior to positioning using the handles 200.

The article 20 is illustrated in FIG. 1 in a flat, uncontracted state (i.e., without elastic induced contraction). The garment-facing surface 120 of the article 20 is facing the viewer. The article 20 includes a longitudinal centerline 100 and a lateral centerline 110. The article 20 may comprise a chassis 22. The article 20 and chassis 22 are shown to have a front waist region 36, a rear waist region 38 opposed to the front waist region 36, and a crotch region 37 located between the front waist region 36 and the rear waist region 38. The waist regions 36 and 38 generally comprise those portions of the article 20 which, when worn, encircle the waist of the wearer. The waist regions 36 and 38 may include elastic elements such that they gather about the waist of the wearer to provide improved fit and containment. The crotch region 37 is that portion of the article 20 which, when the article 20 is worn, is generally positioned between the legs of the wearer.

The outer periphery of chassis 22 is defined by longitudinal side edges 12 and end edges 14. The chassis 22 may have opposing longitudinal side edges 12 that are oriented generally parallel to the longitudinal centerline 100. However, for better fit, longitudinal side edges 12 may be curved or angled to produce, for example, an “hourglass” shape diaper when viewed in a plan view. The chassis 22 may have opposing end edges 14 that are oriented generally parallel to the lateral centerline 110.

The chassis 22 may comprises a liquid permeable topsheet 24 having longitudinal side edges 25, a backsheet 26, and an absorbent core 28 between the topsheet 24 and the backsheet 26. The absorbent core 28 may have a body-facing surface and a garment facing-surface. The topsheet 24 may be joined to the core 28 and/or the backsheet 26. The backsheet 26 may be joined to the core 28 and/or the topsheet 24. It should be recognized that other structures, elements, or substrates may be positioned between the core 28 and the topsheet 24 and/or backsheet 26. In certain embodiments, the chassis 22 comprises the main structure of the article 20 with other features may added to form the composite article structure. While the topsheet 24, the backsheet 26, and the absorbent core 28 may be assembled in a variety of well-known configurations, exemplary configurations are described generally in U.S. Pat. Nos. 3,860,003; 5,151,092; 5,221,274; 5,554,145; 5,569,234; 5,580,411; and 6,004,306.

The topsheet 24 is generally a portion of the article 20 that may be positioned at least in partial contact or close proximity to a wearer. Suitable topsheets 24 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet 24 is generally supple, soft feeling, and non-irritating to a wearer's skin. Generally, at least a portion of the topsheet 24 is liquid pervious, permitting liquid to readily penetrate through the thickness of the topsheet 24. A particularly preferred topsheet 24 is available from BBA Fiberweb, Brentwood, Tenn.. as supplier code 055SLPV09U.

Any portion of the topsheet 24 may be coated with a lotion as is known in the art. Examples of suitable lotions include those described in U.S. Pat. Nos. 5,607,760; 5,609,587; 5,635,191; and 5,643,588. The topsheet 24 may be fully or partially elasticized or may be foreshortened so as to provide a void space between the topsheet 24 and the core 28. Exemplary structures including elasticized or foreshortened topsheets are described in more detail in U.S. Pat. Nos. 4,892,536; 4,990,147; 5,037,416; and 5,269,775.

The absorbent core 28 may comprise a wide variety of liquid-absorbent materials commonly used in disposable diapers and other absorbent articles. Examples of suitable absorbent materials include comminuted wood pulp, which is generally referred to as air felt creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. These materials may be combined to provide a core 28 in the form of one or more layers (individual layers not shown) that may include fluid handling layers such as acquisition layers, distribution layers and storage layers. Such absorbent cores 28 may also include layers (not shown) to stabilize other core components. Such layers include a core cover and a dusting layer. A suitable material for such layers is a spunbonded/meltblown/spunbonded nonwoven having a basis weight between about 10 and 15 gsm (the meltblown layer comprises <5 gsm) as is available from Avgol America, Inc. of Knoxville, N.C. For example, Exemplary absorbent structures for use as the absorbent core 28 are described in U.S. Pat. Nos. 4,610,678; 4,673,402; 4,834,735; 4,888,231; 5,137,537; 5,147,345; 5,342,338; 5,260,345; 5,387,207; 5,397,316; and 5,625,222 and in published U.S. patent application Ser. Nos. 04/0162536 and 04/0167486.

The backsheet 26 is generally positioned such that it may be at least a portion of the garment-facing surface 120 of the article 20. Backsheet 26 may be designed to prevent the exudates absorbed by and contained within the article 20 from soiling articles that may contact the article 20, such as bed sheets and undergarments. In certain embodiments, the backsheet 26 is substantially water-impermeable. Suitable backsheet 26 materials include films such as those manufactured by Tredegar Industries Inc. of Terre Haute, Ind. and sold under the trade names X15306, X10962, and X10964. Other suitable backsheet 26 materials may include breathable materials that permit vapors to escape from the article 20 while still preventing exudates from passing through the backsheet 26. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746 and U.S. Pat. No. 5,865,823. Other breathable backsheets including nonwoven webs and apertured formed films are described in U.S. Pat. No. 5,571,096. An exemplary, suitable backsheet is disclosed in U.S. Pat. No. 6,107,537. Other suitable materials and/or manufacturing techniques may be used to provide a suitable backsheet 26 including, but not limited to, surface treatments, particular film selections and processing, particular filament selections and processing, etc.

Backsheet 26 may also consist of more than one layer, as illustrated in the cut-away of FIG. 1. The backsheet 26 may comprise an outer cover 26a and an inner layer 26b. The outer cover 26a may have longitudinal side edges 27a and the inner layer 26b may have longitudinal side edges 27b. The outer cover 26a may be made of a soft, non-woven material. The inner layer 26b may be made of a substantially water-impermeable film. The outer cover 26a and an inner layer 26b may be joined together by adhesive or any other suitable material or method. A particularly suitable outer cover 26a is available from Corovin GmbH, Peine, Germany as supplier code A18AH0, and a particularly suitable inner layer 26b is available from RKW Gronau GmbH, Gronau, Germany as supplier code PGBR4WPR. While a variety of backsheet configurations are contemplated herein, it would be obvious to those skilled in the art that various other changes and modifications can be made without departing from the spirit and scope of the invention.

The article 20 may include front ears 40 and back ears 42. The front and/or back ears 40, 42 may be unitary elements of the article 20 (i.e., they are not separately manipulative elements secured to the article 20, but rather are formed from and are extensions of one or more of the various layers of the article). In certain embodiments, the front and/or back ears 40, 42 may be discrete elements that are joined to the chassis 22, as shown in FIG. 1. Discrete front and/or back ears 40, 42 may be joined to the chassis 22 by any bonding method known in the art such as adhesive bonding, pressure bonding, heat bonding, and the like. In other embodiments, the front and/or back ears 40, 42 may comprise a discrete element joined to the chassis 22 with the chassis 22 having a layer, element, or substrate that extends over the front and/or back ear 40, 42. The front ears 40 and back ears 42 may be extensible, inextensible, elastic, or inelastic. The front ears 40 and back ears 42 may be formed from nonwoven webs, woven webs, knitted fabrics, polymeric and elastomeric films, apertured films, sponges, foams, scrims, and combinations and laminates thereof. In certain embodiments the front ears 40 and back ears 42 may be formed of a nonwoven/elastomeric material laminate or a nonwoven/elastomeric material/nonwoven laminate. A suitable elastic back ear 42 may be a laminate comprising an elastomeric film (such as is available from Tredegar Corp, Richmond, Va., as supplier code X25007) disposed between two nonwoven layers (such as is available from BBA Fiberweb, Brentwood, Tenn.. as supplier code FPN332). While the following embodiments are directed to back ear 42 design and construction, these embodiments are equally applicable to front ear 40 design and construction. It should be recognized that any combination of the following embodiments may be used for the back ear 42 and/or the front ear 40.

The article 20 may also include a fastening system 50. When fastened, the fastening system 50 interconnects the front waist region 36 and the rear waist region 38 resulting in a waist opening with a waist circumference that may encircle the wearer during wear of the article 20, and provides leg openings through which the wearer's legs may depend. The fastening system may interconnect the front and rear waist regions 36, 38 by any suitable technique including, but not limited to, joining together portions of the article using refastenable (tape tabs, hook and loop fastening components, interlocking fasteners such as tabs & slots, buckles, buttons, snaps, and/or hermaphroditic fastening components, cohesive bond, etc.) and/or non-refastenable bonds (e.g., seam, weld, adhesive, etc.).

Thus, FIG. 1 illustrates a fastening system 50 having an engaging member 52 and a receiving member 54. Further, according to one embodiment, the engaging member 52 has an engaging surface 53 that may comprise hooks, loops, an adhesive, a cohesive, or other fastening member, and the receiving member 54 may have a surface that allows for engagement of the engaging member 52. For example, the receiving member 54 may comprise hooks, loops, an adhesive, a cohesive, or other fastening component that can receive the engaging member 52. Suitable engaging member 52 and receiving member 54 combinations include but are not limited to hooks/loop, hooks/hooks, adhesive/polymeric film; cohesive/cohesive, adhesive/adhesive; tab/slot; and button/button hole. The fastening system 50 may comprises a fastener such as, although any other known fastening means are generally acceptable. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098.

In alternative embodiments, the article 20 may be preformed by the manufacturer to create a pant. A pant may be preformed by any suitable technique including, but not limited to, joining together portions of the article using refastenable and/or non-refastenable bonds (e.g., seam, weld, adhesive, cohesive bond, fastener, etc.). For example, the article 20 of FIG. 1 may be manufactured with the fastening system 50 engaged (i.e., the engaging member 52 is joined to the receiving member 54). In this fashion, where a pair of front ears 40 and a pair of rear ears 42 have been provided (as illustrated), one each of the front ears 40 may be fastened to one each of the rear ears 42 to define a pair of joined front and rear ears 40, 42 (as also illustrated).

According to a further embodiment, the article 20 of FIG. 1 may be manufactured with the front ears 40 joined to the back ears 42 by way of a bond such as an adhesive bond, a mechanical bond, or some other bonding technique known in the art. Suitable pants are disclosed in U.S. Pat. Nos. 5,246,433; 5,569,234; 6,120,487; 6,120,489; 4,940,464; 5,092,861; 5,897,545; and 5,957,908.

The fastening system 50 may also provide a means for holding the article in a disposal configuration as disclosed in U.S. Pat. No. 4,963,140. The fastening system 50 may also include primary and secondary fastening systems, as disclosed in U.S. Pat. No. 4,699,622. The fastening system 50 may be constructed to reduce shifting of overlapped portions or to improve fit as disclosed in U.S. Pat. Nos. 5,242,436; 5,499,978; 5,507,736; and 5,591,152.

The article 20 may include barrier cuffs 60 and/or gasketing cuffs 70. Gasketing cuffs 70 may also be referred to as outer leg cuffs, leg bands, side flaps, leg cuffs, or elastic cuffs. Barrier cuffs 60 may also be referred to as second cuffs, inner leg cuffs or “stand-up” elasticized flaps.

The gasketing cuff 70 may be substantially inelastic or may be elastically extensible to dynamically fit at the wearer's leg. The gasketing cuff 70 may be formed by one or more elastic members 72 (such as elastic strands) operatively joined to the topsheet 24, backsheet 26, or any other suitable substrate used in the formation of the article 20. Suitable gasketing cuff construction is further described in U.S. Pat. No. 3,860,003.

The barrier cuff 60 may have a distal edge 61 and a proximal edge 63 that run substantially parallel to the longitudinal centerline 100. The barrier cuff 60 may span the entire longitudinal length of the article 20. The barrier cuff 60 may be formed by a flap 62 and an elastic member 64 (such as elastic strands). The flap 62 may be a continuous extension of any of the existing materials or elements that form the article 20. In other embodiments, such as shown in FIG. 1, the barrier cuff 60 may be a discrete element. In such embodiments, the barrier cuff 60 comprising the flap 62 and the elastic member 64 may be formed then joined to the chassis 22 by a bond 65.

The flap 62 may comprise a variety of substrates such as plastic films and woven or nonwoven webs of natural fibers (e.g., wood or cotton fibers), synthetic fibers (e.g., polyester or polypropylene fibers), or a combination of natural and synthetic fibers. In certain embodiments, the flap 62 may comprise a nonwoven web such as spunbond webs, meltblown webs, carded webs, and combinations thereof (e.g., spunbond-meltblown composites and variants). Laminates of the aforementioned substrates may also be used to form the flap 62. A particularly suitable flap may comprise a nonwoven available from BBA Fiberweb, Brentwood, Tenn.. as supplier code 30926. A particularly suitable elastic member is available from Invista, Wichita, Kans. as supplier code T262P. Further description of diapers having barrier cuffs and suitable construction of such barrier cuffs may be found in U.S. Pat. Nos. 4,808,178 and 4,909,803. The elastic member 64 generally spans the longitudinal length of the barrier cuff 60. In other embodiments, the elastic member 64 may span at least the longitudinal length of the barrier cuff 60 within the crotch region 37. It is desirable that the elastic member 64 exhibits sufficient elasticity such that the proximal edge 63 of the barrier cuff 60 remains in contact with the wearer during normal wear, thereby enhancing the barrier properties of the barrier cuff 60. The elastic member 64 may be connected to the flap 62 at opposing longitudinal ends. In certain embodiments, the flap 62 may be folded over onto itself so as to encircle the elastic member 64. A bond 67 may be used to secure the folded section of the flap 62.

The barrier cuffs 60 and/or gasketing cuffs 70 may be treated, in full or in part, with a lotion, as described above with regard to topsheets, or may be fully or partially coated with a hydrophobic surface coating as detailed in U.S. application Ser. No. 11/055,743, which was filed Feb. 10, 2005.

The article 20 may also include at least one handle 200 joined to the chassis 22, the handle 200 assisting in the application of the article 20 onto a wearer or removal of the article from a wearer. The handle 200 is designed to deflect upon use from an original position, but also to withstand the forces necessary to apply or remove the article 20, including, for example, insertion of the wearer's feet through the leg openings, elevating the article up the wearer's legs and over the wearer's buttocks, achieving an ideal snug fit, and lowering the article 20. The handle 200 is also designed to return to the original shape some time after the handle is used to apply or remove the article 20.

In particular, the handle 200 may preferably be of a size and shape that assist the user (whether child or caregiver) in using the handle 200 to position the article 20 on the wearer. The handle 200 may particular facilitate use by persons (such as children) with limited motor skills and/or dexterity. As the handle 200 deforms, the handle 200 may actually facilitate the user grasping the handle to a greater degree than when the user initially grasps the handle 200. During use, the handle 200 is capable of transmitting a considerable amount (at least 30-50%) of the force applied to the handle with minimal strain, such that the absolute force transmitted by the handle 200 to the chassis 22 may be at least about 20N. Further, after the handle 200 has been released, the handle 200 should recover to substantially or nearly substantially its initial shape, such that the degree to which the handle 200 interferes with the wearer or the wearer's clothing is limited.

One way in which this may be achieved is by forming the handle 200 of a slow recovery elastomer, which elastomer is discussed in greater detail below. The slow recovery elastomer may exhibit a normalized unload force of at least about 0.04 N/mm² at 37C at 60% strain after 5 minutes hold time as measured by the Two Cycle Hysteresis Test, described in greater detail below. The slow recovery elastomer may also exhibit a post elongation strain of 60% or greater after 15 seconds of recovery at 22C as measured by the Post Elongation Recovery Test, also described in greater detail below. In particular, the slow recovery elastomer may be prepared from a composition comprising an elastomeric polymer, optionally at least one modifying resin, and optionally one or more additives.

Further, the elastomer may be used in composite structure, such as a laminate, for example. That is the elastomer, in the form of an elastomeric film, may be joined to one or more layers of a nonwoven material, for example. If two nonwoven layers are used, the elastomer film may be disposed between the nonwoven layers, and joined (discretely or continuously) thereto through the use of an adhesive, for example, applied to both sides of the elastomeric film. The nonwoven material may be available from BBA Nonwovens of Nashville, Tenn.. under the designation “Highly Elongatable Carded Nonwoven.” Further, the adhesive may be available from Ato Findley of Milwaukee, Wis. under the designation H2031. The adhesive may be applied in a spiral pattern, as illustrated in U.S. Pat. No. 3,991,173, for example, at a basis weight of about 0.116 gsm. Alternatively, the layers may be bonded by heat bonding, ultrasonic bonding, pressure bonding, dynamic mechanical bonding, or any other method known in the art.

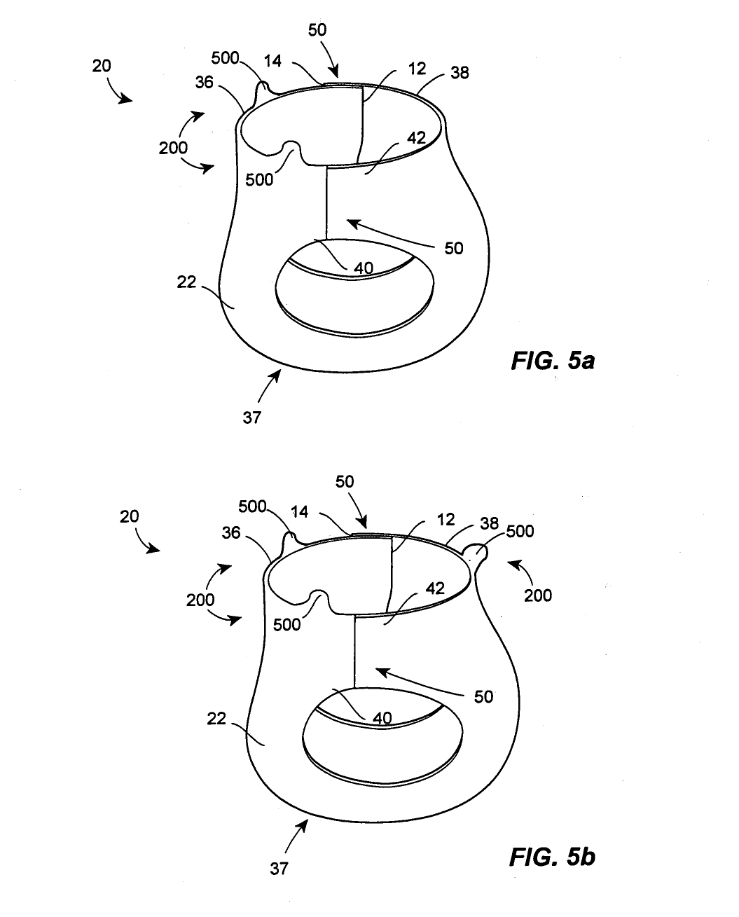

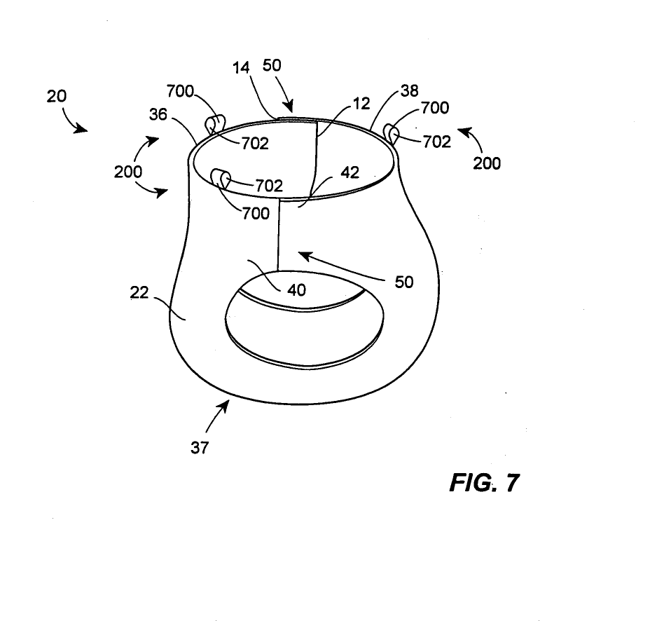

As for the shape and dimensions of the handles 200, these features may vary considerably. Nor is placement of the handles 200 confined to any particular region of the article 20, but may span any portion of the article 20, including circumscribing the entire article 20. It will thus be recognized that the embodiments illustrated in FIGS. 4, 5a-b, 6a-d, 7, 8a-b, and 9a-b are exemplary, not exhaustive. It will also be recognized that the use of handles 200 of different embodiments in combination (e.g., one of the tabs from FIGS. 5a, 5b with one of the loops of FIG. 7) is also within the scope of the present disclosure.

Thus, FIG. 2 illustrates a first embodiment of the handle 200. According to this embodiment, the handles 200 may include a pair of flaps 210 (only one of which is shown) that depend from the chassis 22. During application or removal, the fingers of the wearer or caregiver may be inserted, according to the embodiments shown herein, under, into, or through said flap 210. While not limited in theory, the flap 210 allows the individual applying or removing the article 20 to cup the flap 210 between the fingers and palm, or encircle the flap 210 by fingers and thumb.

According to the embodiment shown in FIG. 2, the flaps 210 may be located across the ears 40, 42. In particular, the flaps 210 may be defined separately from and joined to the backsheet 26 at longitudinal regions 212, one forward of the ears 40 in the front waist region 36 and another behind the ears 42 in the rear waist region 38. The flaps 210 may be joined to the chassis 22 by an adhesive, heat bonds, pressure bonds, ultrasonic bonds, dynamic mechanical bonds, or combinations thereof, for example, which may also be true of the other structures that define the handles 200 in the other embodiments described herein.

According to the embodiment in FIG. 2, the resulting flap 210 should be of an effective length such that a wearer (or caregiver) may engage the flap 210 with his or her fingers. The effective length of the flap 210, which may be defined from an upper edge 214 to a lower edge 216 of the flap 210, may be from about 10 mm to about 50 mm. In certain embodiments, the effective length may be from about 20 mm to about 30 mm. It will be recognized that selection of the effective length may depend on a number of factors, including the size of the article 20 to which the flap 210 is joined.

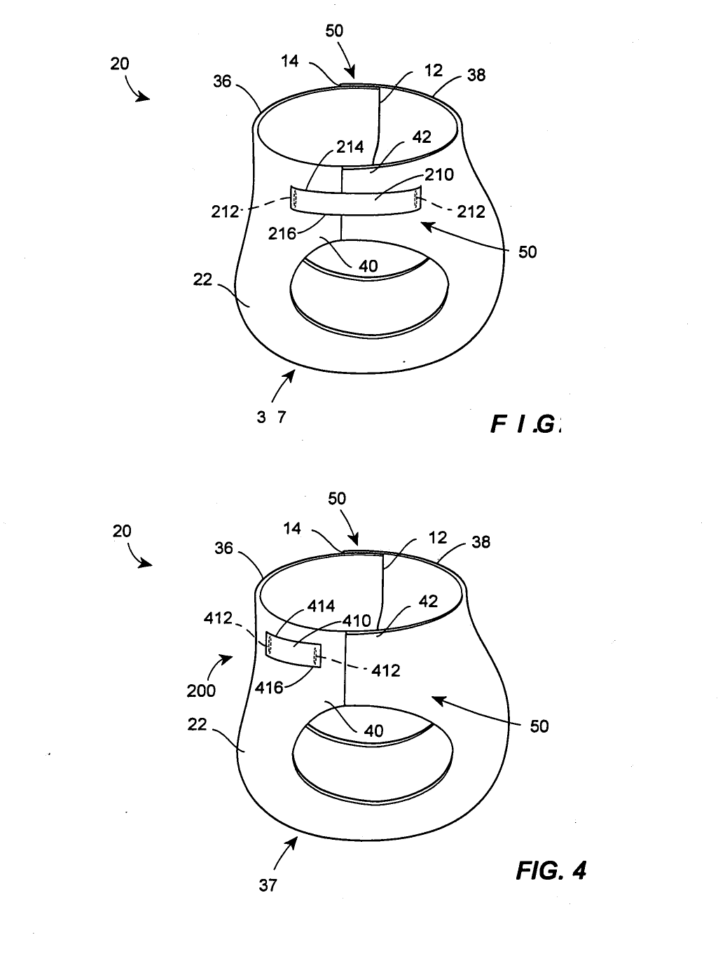

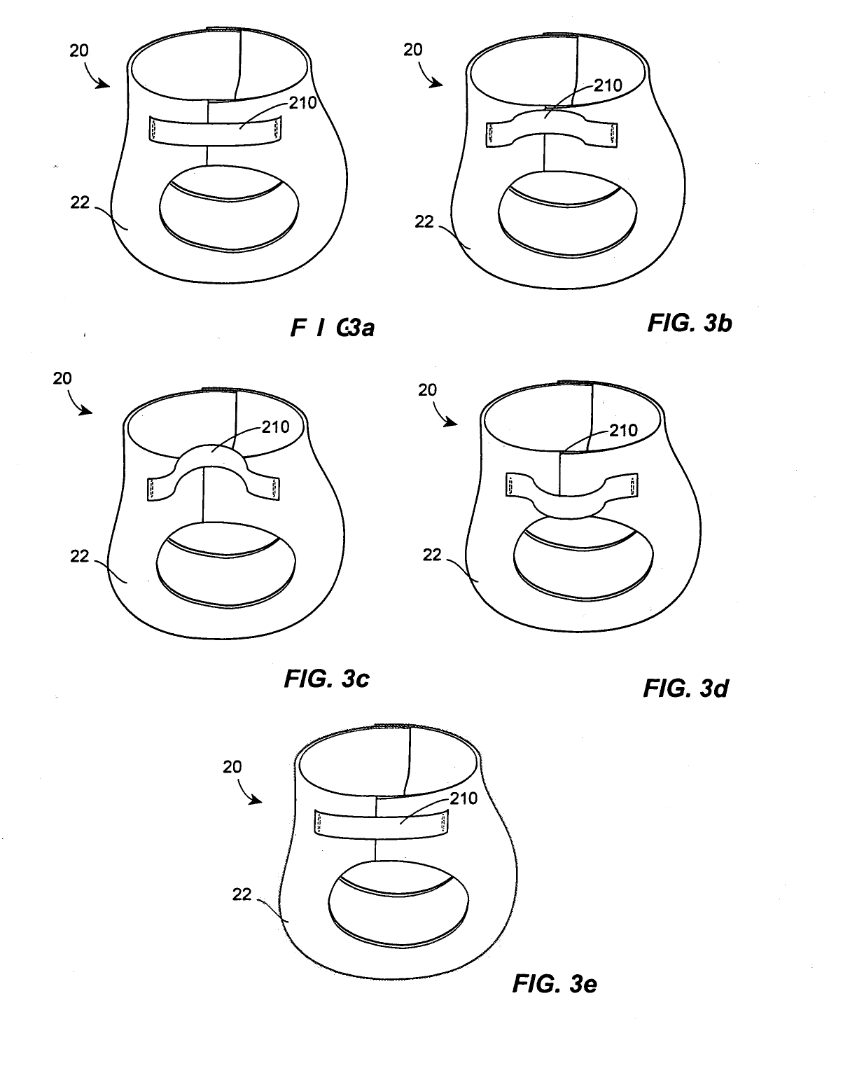

FIGS. 3a-3e show the handles 200 according to the embodiment of FIG. 2 (flaps 210) as they may appear in various positions or states during use of the handles 200. FIG. 3a illustrates the flaps 210 in a first position or a first state, at which time the wearer has disposed his or her legs through the waist opening and the leg openings, but prior to application of any force to the flaps 210. FIG. 3b illustrates the flaps 210 in a second position or state as the user (child or caregiver) grasps the flaps 210 to position the article 20 on a child. Some slight deformation of the flaps 210 may have already occurred, which deformation may make the flaps 210 easier for the user to grasp. This may be especially important for children using the flaps 210 to assist themselves in putting on the absorbent article 20. FIG. 3c illustrates the flaps 210 in a third substantially deformed or nearly substantially deformed position or state in which considerable force may have been applied to the flaps 210. Even in this state, the flaps 210 have sufficient stiffness to transfer a substantial percentage (for example, 30-50%) of the force applied to the flaps 210 to the remainder of the article 20 to move the article 20 upward along the legs toward the child's waist. FIG. 3d illustrates the flaps 210 in a fourth position or state after the child or caregiver has released the flaps 210, for example, after the article 20 has been positioned. In this position or state, the flaps 210 have already begun the process of recovery. FIG. 3e illustrates the flaps 210 in a fifth position or state, after the process of recovery has completed. In this position or state, the length of the flap 210 between the regions 212 may be substantially the same as the original length of the flap 210 between the regions 212 illustrated in FIG. 3a, although according to certain embodiments, the length of the flap 210 in the state of FIG. 3e may remain longer that the length of the flap in the state of FIG. 3a.

FIG. 4 illustrates a second embodiment of the handle 200, similar to that shown in FIGS. 2 and 3a-3e. According to this embodiment, the handles 200 also include a pair of flaps 410 (only one of which is shown) that depend from the chassis 22. During application or removal, the fingers of the wearer or caregiver may be inserted, according to the embodiments shown herein, under, into, or through said flap 410. However, according to the embodiment shown in FIG. 4, the flaps 410 are located in the front waist region 36 forward of the ears 40.

FIGS. 5a and 5b illustrate further embodiments of the handles 200. According to these embodiments, the handle 200 comprises a tab 500 depending from the waist regions 36, 38 away from the crotch region 37. The tabs 500 may be disposed slightly forward of the ears 40 (FIG. 5a, 5b), as well as elsewhere along the waist regions 36, 38 (FIG. 5b). While the placement of the tabs 500, as illustrated, exhibits some form of symmetry, the tabs 500 may be positioned in an asymmetric fashion. According to the embodiment illustrated in FIGS. 5a and 5b, the tabs 500 are defined by a section of slow recovery elastomer that is joined to the chassis 22, for example, the backsheet 26. The tabs 500 may be engaged, for example, by pinching the tab 500 between the fingers of the wearer (or caregiver) and pulled in a longitudinal direction.

As illustrated, two tabs 500 are illustrated in the embodiment of FIG. 5a, while three tabs 500 are illustrated in the embodiment of FIG. 5b, although a larger or a smaller number of tabs 500 may also have been included. According to both embodiments, two of the tabs 500 are in the front waist region 36 just forward of the ears 40. In the embodiment of FIG. 5b, an additional tab is joined to the backsheet 26 in the rear waist region 38 centrally between the ears 42. The tabs 500 may be used singly or in combination to assist the wearer (or caregiver) positioning the article 20 on the wearer.

For example, according to the embodiment of FIG. 5b, the wearer (or caregiver) may grip the two tabs 500 in the front waist region 36. Alternatively, the wearer (or caregiver) may grip or one of the tabs 500 in the front waist region 36 and the tab 500 in the rear waist region 38. As a further alternative, the wearer (or caregiver) may grip just the tab 500 in the rear waist region 38 alone. Certain combinations may be used when the wearer places his or her feet through the leg openings, for example, while others combinations may be used for the final fitting of the article 20 in place snuggly on the wearer.

The dimensions of the tabs 500 may be of an effective size such that, as explained above, a wearer (or caregiver) can engage the tab 500 by pinching the tab 500 between his or her fingers. It will be recognized that the maximum dimensions of the tab 500 may be limited by the desire to have the article 20 maintain a comfortable fit and to limit interfere with the application of other outer garments (i.e., pants, gown). Ideally, the tab 500 may have an effective length (measured between the end edge 14 and the longitudinally extreme edge of the tab 500) of about 10 mm to about 50 mm. Most preferably, the tab 500 has a length of about 20 mm to about 30 mm.

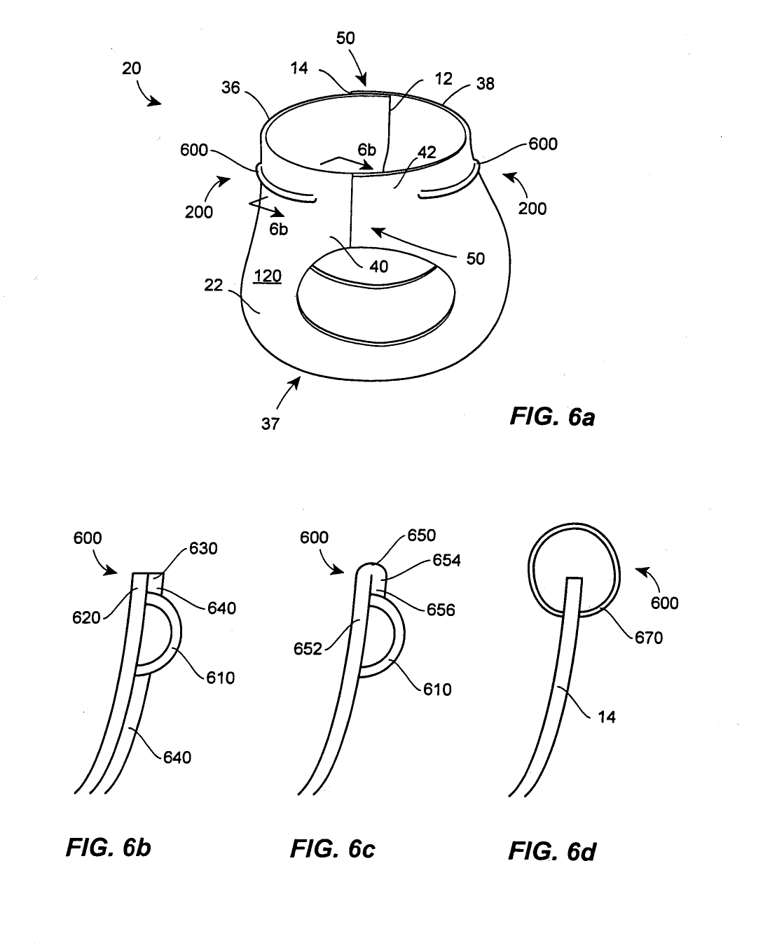

Still further embodiments are illustrated in FIGS. 6a-d. According to these embodiments, the handle 200 is defined by a ridge 600 protruding from the chassis 22 of the article 20. The ridge 600 may be a protrusion joined to and/or depending from the garment-facing surface 120 of the backsheet 26. As also illustrated, the ridge 600 may be located in the waist regions 36, 38 proximate to or at the end edges 14. Furthermore, the ridge 600 may fully, partially, or intermittently circumscribe the article 20. Thus, while partially circumferential peripheral ridges are illustrated, the ridge 600 will serve the same functional purpose even where fully or intermittently circumscribing the article 20. The ridge 600 may have a minimum effective cross-dimension such that the ridge 600 provides increased caliper (i.e., a linear dimension as measured orthogonal to the plane defined by the longitudinal and lateral axes) which may be engaged or gripped by the wearer's (or caregiver's) fingers and pulled in a longitudinal direction.

Turning first to the embodiment of FIGS. 6a and 6b, the ridge 600 comprises an insert 610 that may have has a relatively semi-circular cross-sectional dimension, and may be made of the slow recovery elastomer. The insert 610 may be interposed between two layers, which may be referred to as an inner layer 620 and an outer layer 630. According to one embodiment, the inner layer 26b of the backsheet 26 may define the inner layer 620, the outer layer 26a may define the outer layer 630, and the insert 410 may thus depend from the garment-facing surface 120 of the backsheet 26. Alternatively, the insert 410 may be interposed between, for example, the backsheet 26 and a discrete patch that may be welded to the backsheet 26 adjacent the insert 610. According to the embodiment illustrated in FIGS. 6a and 6b, the layers 620, 630 may be welded together at the contact points 640 immediately adjacent to the insert 610 to limit shifting of the insert relative to the layers 620, 630. Optionally, the insert 610 may be welded to one or more layers 620, 630.

It will be recognized that it is not necessary to interpose the insert 610 between two layers; a single layer 650 may suffice. Thus, it will be seen that in the embodiment illustrated FIG. 6c, the insert 610 is interposed between the single layer 650, which layer 650 may have a first region 652 and a second region 654 that is folded over or around the insert 610. In such a configuration, the single layer 650 is joined to itself (by welding, for example) at contact points 656 immediately adjacent to the insert 610 to limit relative movement or shifting of the insert 610. It should be noted that a single contact point 656 may exist especially if the insert 610 is disposed immediately adjacent to the end edge 14.

As still another alternative embodiment, the ridge 600 may be affixed to the backsheet 26 of the article 20 without any overlapping layer. For example, FIG. 6d illustrates a cap 670 along the end edge 14 of the backsheet 26, and made of the slow recovery elastomer. The cap 670 has a substantially circular cross-section, discontinuous only where the cap 670 is joined to the chassis 22. The cap 670 may be joined along the end edge 14 (as shown), or may be joined to the garment-facing surface 120 immediately adjacent the end edge 14, the wearer-facing surface immediately adjacent the end edge 14, and combinations thereof and therebetween.

The maximum effective cross-dimension of the ridge 600 may vary, while recognizing that it may be desired to maintain a comfortable fit for the article 20 and to limit interference with the application of other outer garments (i.e., prohibit self-application of pants, gown, etc.). For example, the ridge 600 may have an effective cross-dimension of about 2 mm to about 15 mm. According to certain embodiments, the ridge 600 has an effective cross-dimension of about 5 mm to about 8 mm. The effective cross-dimension may be described as the difference between the maximum caliper of article 20 at the ridge 600 and the minimum caliper of the article 20 as measured immediately adjacent the ridge 600.

FIG. 7 illustrates yet another embodiment of the handle 200. As shown in FIG. 7, the handle 200 includes a trio of loops 700. The loops 700 may be formed from a single piece of slow recovery elastomer, with the distal ends of the piece of material folded back onto themselves and joined together. The resulting structure may then be joined to the chassis. Alternately, the welding of the distal ends of the loop 700 and the joining of the loop 700 to the article 20 may be performed in a single step by any method known in the art (e.g., welding). As illustrated, the loops 700 are disposed slightly forward of the ears 40 in the front waist region 36, and centrally between the ears 42 in the rear waist region 38. The loops 700 may be grasped by the wearer or caregiver, with the fingers potentially being disposed in or through the opening 702 defined by the wall of the loop 700.

The position and joining of the loops 700 may vary. For example, according to alternative embodiments of the invention, the loops 700 may be disposed elsewhere on the article 20. For example, similar to the embodiment shown in FIG. 5b, two loops 700 may be disposed in the front waist region 38, or a single loop 700 may be disposed in the rear waist region 38. The loop 700 may be joined to the chassis 22 by disposing one or both of the distal ends between the topsheet 22 and backsheet 26. Alternatively, the distal end(s) of the loop 700 may be attached to the topsheet 22 or the backsheet 26 at or near the end edges 14, or the topsheet 22 or backsheet 26 may have a region adjacent to the end edges 14, for example, that is constructed from the slow recovery elastomer.

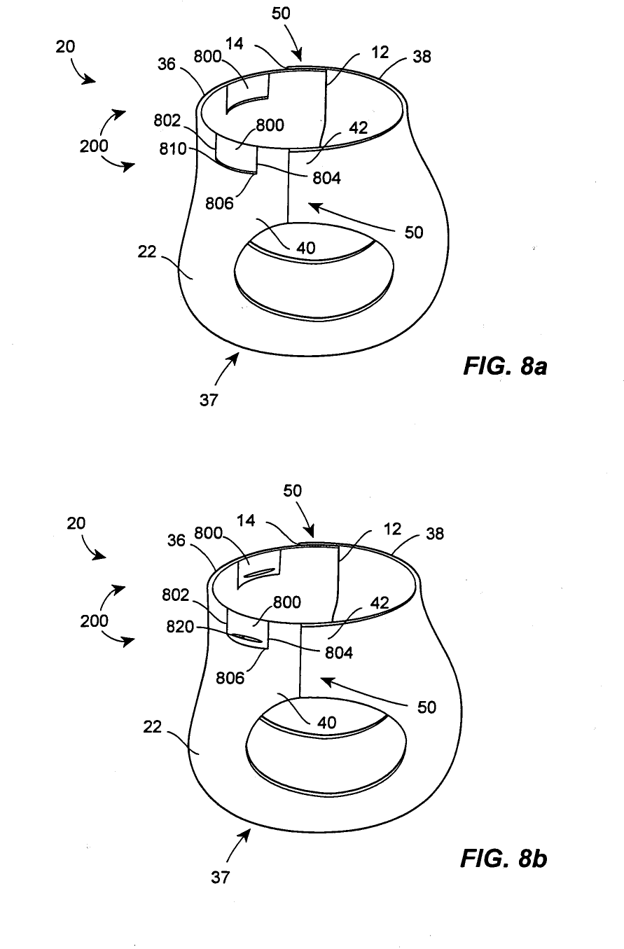

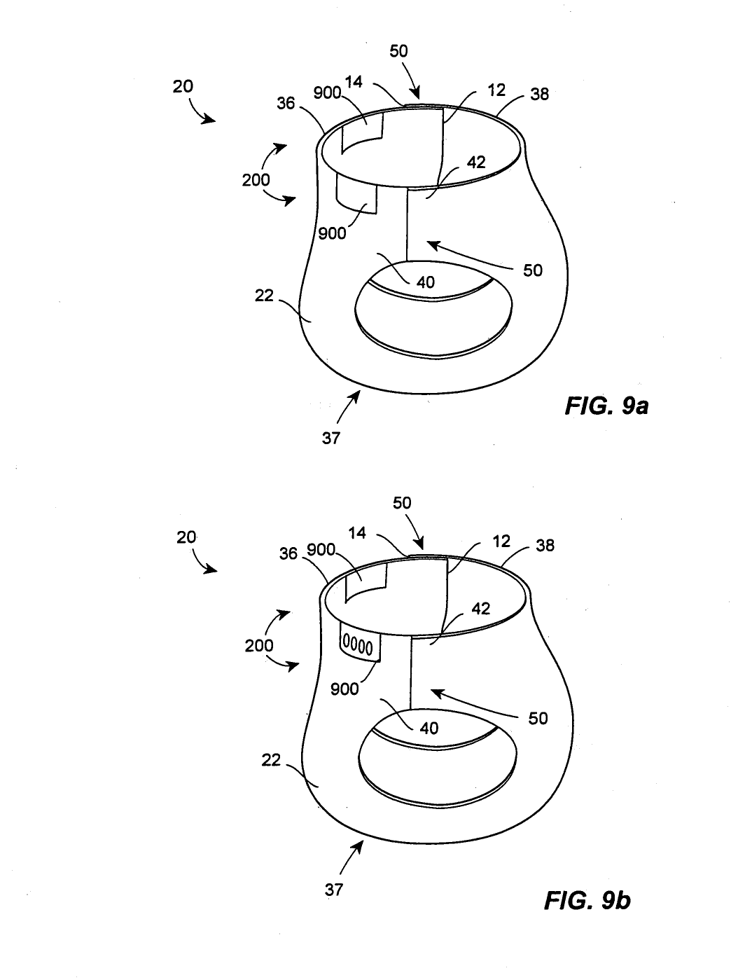

FIGS. 8a, 8b, 9a, and 9b illustrate embodiments wherein the handle 200 is defined by one or more sections of slow recovery elastomer that defines a region of the article 20 that does not depend from or extend beyond typical dimensions for the article. Stated another way, the section of slow recovery material is joined to the article 20 in such a fashion that the region of the article 20 so defined does not appear to differ in longitudinal and/or lateral dimension and/or thickness than a typical article 20 of similar structure that does not include handles 200, other than openings or slits may be provided to facilitate use of the handles 200.

Consequently, FIGS. 8a and 8b illustrate embodiments of the handle 200 wherein a region 800 of the article 20 is defined by a section of the slow recovery elastomer, which region 800 either includes an opening or aperture therein or an opening at the boundary thereof. In both of the embodiments illustrated, the handles 200 are defined by a region 800 that is slightly forward of the ears 40 in the waist regions 36 of the article 20, although it will recognized that the regions 800 may have been defined at the rear and/or the front of the article 20 in the alternative. The sections of slow recovery elastomer that defines the regions 800 may be joined along longitudinal edges 802, 804 to either the topsheet 22 or the backsheet 26, or interposed and joined between the topsheet 22 and the backsheet 26. The regions 800 may also be partially or totally (FIG. 8b) joined to an edge of the ears 40, 42.

According to the embodiment of FIG. 8a, the region 800 is attached at ends 802, 804 to the backsheet 26, and not along the interface 806 between the region 800 and the ears 40, 42. The interface 806 thus defines an opening or aperture in the form of a substantially linear slit 810. The wearer can insert his or her fingers through the slit 810, grasp the region 800, and apply or remove the article 20.

According to the embodiment of FIG. 8b, the region is attached at ends 802, 804 to the backsheet 26, and along the interface 806 between the region 800 and the ears 40, 42. An opening or aperture in the form of a hole 820 (i.e., a void with a non-negligible area) may be defined in the region 800. Such a hole 820 would operate in a manner similar to the slit 810, while being more conspicuous to the wearer (or caregiver). The hole 820 may be elliptical as illustrate, although other shapes may be used as well.

FIGS. 9a and 9b illustrate embodiments of a handle 200 defined by a region 900 of slow recovery material with a distinct grippable surface characteristic in whole (FIG. 9a) or in part (FIG. 9b). The distinct grippable surface characteristic may result from a palpable difference in texture, pliancy, tackiness, or friction as compared to the surface immediately adjacent the region 900. This difference may be imparted to the region 900 by a variety of methods.

As one example, consider a distinct grippable surface characteristic resulting from a palpable difference in friction. Generally, the backsheet 26 may have a coefficient of static friction of about 0.15 to about 0.23. Coefficient of static friction values at these relatively low levels facilitate application of garments over the article 20. According to the present disclosure, the region 900 may have a distinct grippable surface characteristic exhibiting a coefficient of static friction from greater than about 0.3 to less than about 4.0, such as between about 0.4 and about 2.0, or between about 0.7 and about 1.5. The coefficient of static friction may be measured according to the test method disclosed in U.S. Pat. No. 6,626,879.

According to an embodiment, the characteristic can be imparted to the region 900 by joining a material that exhibits the distinct grippable surface characteristic to the region 900, by coating or in patches, for example. In regard to the former example, where the region 900 is joined to the material by coating, coatings of pressure-sensitive materials or of tacky materials may be used to increase the coefficient of static friction. Such coatings may include, but are not limited to, ethylene vinyl acetate copolymers, polyvinyl acetate, styrene-butadiene, cellulose acetate butyrate, ethyl cellulose, acrylics, synthetic rubber hot melt, and other hot melts. The methods for coating may include, but are not limited to, extrusion, coating, slot coating, gravure printing, and screen printing. In regard to the latter example, where the region 900 is joined to patches, the patches may be made from a number of different materials that are thin, flexible, and that can be affixed to the region. Examples of materials from which such patches can be made may include, but are not limited to, polymeric films, apertured films, fibrous nonwoven sheets, scrims, scrim nettings, or fibrous flocked substrates. The patches may be affixed to the region by affixation means well known in the art, such as heat/pressure affixation. A more detailed discussion of lamination by coating or patches is contained in above-referred U.S. Pat. No. 6,626,879, particularly the disclosure on retention zones.

Additional surface characteristics of the region 900 instead of and in addition to an increased friction coefficient may be included. For example, the region 900 may exhibit a texture such that the material comprising the region has a palpable quality. For example, texture can be imparted mechanically by ring rolling the region 900. An exemplary method of ring rolling is illustrated in U.S. Pat. No. 6,383,431. The region 900 can exhibit pliancy such that the material comprising the region yields to touch. The region 900 can exhibit tackiness such that the material comprising the region has a slightly adhesive or gummy feel to the touch.

An absorbent article 20 comprising one or more of the handle embodiments previously presented may be packaged in a kit containing a plurality of the absorbent articles 20. The absorbent articles 20 are positioned within the kit such that the handle 200 is readily accessible. Readily accessible may refer to the fact that the handle 200 is visible to the wearer or caregiver and that the handle 200 may be grasped and used for removal of the absorbent article 20 from the kit. The kit may require activation so that the article 20 becomes accessible (e.g., opening of a lid, removal of a panel, etc.). In a preferred embodiment, the kit is defined by numerous absorbent articles 20 bound together as an entity and covered by a thermoplastic film over wrap. A particularly preferred kit is represented in U.S. Pat. No. 5,934,470. This kit enables absorbent articles 20 to be delivered to and purchased by a consumer while economizing space. The thermoplastic film cover ideally contains an opening means to allow removal of a portion of the thermoplastic film cover and access to the absorbent articles. A typical opening means includes a substantially continuous line of weakness, preferably perforations within the thermoplastic film cover. An exemplary opening means is presented in U.S. Pat. No. 5,036,978.

Upon activation of the opening means, the absorbent articles 20 may be presented individually or multiply for removal from the kit. Regardless of such presentation, the handle 200 may be visible and graspable. The handle 200 may be presented by the manner in which the kit is opened. The handle 200 may be presented in a manner in which the article 20 is folded and/or stacked within the kit. The handle 200 may be presented by the mere size, shape, or position of the handle 200. Typically, absorbent articles 20 are positioned in a uniform manner within the kit (i.e., the articles 20 are folded similarly and bound uniformly) with the waist region 36, 38 being presented. In such a case, a handle 200 as a tab 500 (see FIGS. 5a and 5b) would be readily accessible to a wearer (or caregiver). The tab 500 may be grasped and used to remove the article 20 from the kits. Subsequently, a second article 20 having a tab 500 would be presented from the kit.

### Slow Recovery Elastomer and Testing

As noted above, the handles 200 of the absorbent article 20 may comprise a slow recovery elastomer. The slow recovery elastomer may be used elsewhere within the absorbent article 20, but according to the present disclosure, the slow recovery elastomer is particularly used for the handles. As such, the slow recovery elastomer may be joined to another material or substrate (such as a polymeric film, a nonwoven, a woven, or a scrim) and used in the absorbent article 20.

As mentioned above, the slow recovery elastomer may be prepared from a composition comprising an elastomeric polymer, optionally at least one modifying resin, and optionally one or more additives. The slow recovery elastomer exhibits a normalized unload force of at least about 0.04 N/mm² at 37C at 60% strain after 5 minute hold time as measured by the Two Cycle Hysteresis Test, described in greater detail below. The slow recovery elastomer exhibits a post elongation strain of 30% or greater after 15 seconds of recovery at 22C as measured by the Post Elongation Recovery Test, also described in greater detail below.

A number of elastomeric polymers may be used to prepare the slow recovery elastomer with the requisite normalized unload force and post elongation strain. Elastomeric polymers include, but are not limited to, homopolymers (e.g., crosslinked poly(isoprene)), block copolymers, random copolymers, alternating copolymers, and graft copolymers. Suitable elastomeric polymers comprise styrenic block copolymers, natural and synthetic rubbers, polyisoprene, neoprene, polyurethanes, silicone rubbers, hydrocarbon elastomers, ionomers, and the like.

In one embodiment, the elastomeric polymer may be a block copolymer. A number of block copolymers may be used to prepare the slow recovery elastomer including multi-block, tapered block and star block copolymers. Generally, the block copolymers suitable for use in the slow recovery elastomer may exhibit both elastomeric and thermoplastic characteristics. In such block copolymers a hard block (or segment) may have a glass transition temperature (Tg) greater than about 25C or is crystalline or semicrystalline with a melting temperature (Tm) above about 25C. Preferably, the hard block has a Tg greater than about 35C or is crystalline or semicrystalline with a Tm above about 35C. The hard block portion is typically derived from vinyl monomers including vinyl arenes such as styrene and alpha-methyl-styrene or combinations thereof.

Glass transition temperatures referred to herein are determined by tensile dynamic mechanical analysis performed in the linear elastic region of the material at a frequency of 1 Hz using a temperature ramp method. Suitably, film samples with a uniform thickness of about 0.3 mm or less may be used with a temperature ramp rate of about 1C/min or slower. The TanΔ peak temperature is taken as the Tg of the particular material or phase.

Crystalline melting temperatures referred to herein are determined by Differential Scanning Calorimetry using a temperature ramp rate of 10C/min. The melting endotherm peak temperature is taken as the Tm of the particular crystalline region.

The block copolymers may comprise a soft block (or segment). The soft block generally exhibits a sufficiently low glass transition temperature and/or melting temperature so as not to form glassy or crystalline regions at the use temperature of the copolymer. In one embodiment, the use temperature may be between about room temperature (about 22C) and about body temperature (about 37C). However, other use temperatures are feasible and within the scope of this invention. Such soft blocks are generally physically incompatible with the hard blocks and form separate regions, domains, or phases.

The soft block portion may be a polymer derived from conjugated aliphatic diene monomers. Typically, the soft block monomers contain fewer than about 6 carbon atoms. Suitable diene monomers include butadiene, isoprene, and the like. Particularly preferred soft block polymers include poly(butadiene) and poly(isoprene). Furthermore, it is envisioned that the soft block may be modified to tailor the Tg of the soft block. For example, a random copolymer of isoprene and styrene or a graft of styrene onto poly(isoprene) may be used. In such cases, lower amounts of the modifying resin may be used.

Suitable block copolymers for use in this invention may comprise at least one hard block (A) and at least one soft block (B). The block copolymers may have multiple blocks. In a preferred embodiment, the block copolymer may be an A-B-A triblock copolymer, an A-B-A-B tetrablock copolymer, or an A-B-A-B-A pentablock copolymer. Also, useful herein are triblock copolymers having endblocks A and A′, wherein A and A′ may be derived from different vinyl compounds. Also, useful in the present invention are block copolymers having more than one hard block and/or more than one soft block, wherein each hard block may be derived from the same or different monomers and each soft block may be derived from the same or different monomers.

It should be noted that where the copolymer contains residual olefinic double bonds, the copolymer may be partially or fully hydrogenated if desired. Saturation may often yield beneficial effects in the elastomeric properties of the copolymer.

The elastomeric polymer may be used in the slow recovery elastomer in an effective amount so as to achieve the desired normalized unload forces and post elongation strains. The slow recovery elastomer generally may comprise from about 20% to about 70%, preferably about 30% to about 65%, and most preferably about 45% to about 60% of the elastomeric polymer.

Preferred elastomeric polymers include styrene-olefin-styrene triblock copolymers such as styrene-butadiene-styrene (S-B-S), styrene-ethylene/butylene-styrene (S-EB-S), styrene-ethylene/propylene-styrene (S-EP-S), styrene-isoprene-styrene (S-I-S), hydrogenated polystyrene-isoprene/butadiene-styrene (S-EEP-S), and mixtures thereof. The block copolymers may be employed alone or in a blend of block copolymers.

Particularly preferred block copolymers include styrene-butadiene-styrene (S-B-S) and styrene-isoprene-styrene (S-I-S) block copolymers. Such linear block copolymers of styrene-butadiene-styrene (S-B-S) and styrene-isoprene-styrene (S-I-S) are commercially available under the trade designation Vector from Dexco Polymers L.P., Houston, Tex., and under the trade designation Kraton from Kraton Polymers, Houston, Tex.

Various modifying resins may be used in this slow recovery elastomer. Suitable modifying resins should preferably associate or phase mix with the soft blocks of the elastomeric polymer. Modifying resins should have a sufficiently high molecular weight average such that the glass transition temperature of the soft block is increased resulting in an increase of post elongation strain at 22C after 15 seconds of recovery. While not intending to be bound by this theory, it is believed that the modifying resins raise the Tg of the soft phase to the point where molecular relaxation at the in-use temperature is slowed. This is evidenced by a relatively high post elongation strain.

The slow recovery elastomer may comprise the modifying resin in amounts from about 0% to about 60% by weight. Preferably, the composition comprises from about 20% to about 55% and even more preferably from about 35% to about 45% of the modifying resin.

Suitable modifying resins useful herein may have glass transition temperatures ranging from about 60C to about 180C, more preferably from about 70C to about 150C, and more preferably from about 90C to about 130C.

Suitable modifying resins useful herein should preferably be soft block associating. A solubility parameter is useful in determining whether the modifying resin will phase mix with the soft block of the block copolymer. Generally, modifying resins are selected so that the solubility parameter of the modifying resin is similar to the solubility parameter of the soft block phase. Since common soft block phases have solubility parameters from about 7.0 (cal/cm³)^{1/2} to about 9.0 (cal/cm³)^{1/2}, the modifying resins should have similar solubility parameters. For example in the case where the solubility parameter of the soft block phase is about 8 (cal/cm³)^{1/2}, the solubility parameter of the modifying resin should be from about 7.5 (cal/cm³)^{1/2} to about 8.5 (cal/cm³)^{1/2}. The solubility parameters of the modifying resins may also approximate the solubility of the hard block. However, as long as phase mixing of the modifying resin with the soft block exists, hard block phase mixing should not be read as limiting. A list of solubility parameters for common polymers or resins, along with methods for determining or approximating the solubility parameters can be found in the Polymer Handbook, Third Edition; Wiley Interscience; Section VII pages 519-559.

Modifying resins useful herein include, but are not limited to, unhydrogenated C5 hydrocarbon resins or C9 hydrocarbon resins, partially and fully hydrogenated C5 hydrocarbon resins or C9 hydrocarbon resins; cycloaliphatic resins; terpene resins; polystyrene and styrene oligomers; poly(t-butylstyrene) or oligomers thereof; rosin and rosin derivatives; coumarone indenes; polycyclopentadiene and oligomers thereof; polymethylstyrene or oligomers thereof; phenolic resins; indene polymers, oligomers and copolymers; acrylate and methacrylate oligomers, polymers, or copolymers; derivatives thereof; and combinations thereof. Preferably, the resin is selected from the group consisting of the oligomers, polymers and/or copolymers derived from: t-butylstyrene, cyclopentadiene, iso-bornyl methacrylate, methyl methacrylate, isobutyl methacrylate, indene, coumarone, vinylcyclohexane, methylstyrene, and 3,3,5-trimethylcyclohexyl methacrylate. Preferred modifying resins also include alicyclic terpenes, hydrocarbon resins, cycloaliphatic resins, poly-beta-pinene, terpene phenolic resins, and combinations thereof. “C5 hydrocarbon resins” and “C9 hydrocarbon resins” are disclosed in U.S. Pat. No. 6,310,154.

In general, a variety of additives may be employed to yield a slow recovery elastomer with more favorable characteristics. For example, stabilizers, antioxidants, and bacteriostats may be employed to prevent thermal, oxidative, and bio-chemical degradation of the slow recovery elastomer. Generally, the additive or additives may account for about 0.01% to about 60% of the total weight of the slow recovery elastomer. In other embodiments, the composition comprises from about 0.01% to about 25%. In other suitable embodiments, the composition comprises from about 0.01% to about 10% by weight, of additives.

Various stabilizers and antioxidants are well known in the art and include high molecular weight hindered phenols (i.e., phenolic compounds with sterically bulky radicals in proximity to the hydroxyl group), multifunctional phenols (i.e., phenolic compounds with sulfur and phosphorous containing groups), phosphates such as tris-(p-nonylphenyl)-phosphite, hindered amines, and combinations thereof. Representative hindered phenols include t-butylhydroxyquinone; 1,3,5-trimethyl-2,4,6-tris(3-5-di-tert-butyl-4-hydroxybenzyl)benzene; pentaerythritol tetrakis-3(3,5-di-tert-butyl-4-hydroxyphenyl)propionate; n-octadecyl-3(3,5-ditert-butyl-4-hydroxyphenyl)propionate; 4,4′-methylenebis(4-methyl-6-tert butylphenol); 4,4′-thiobis(6-tert-butyl- -o-cresol); 2,6-di-tert-butylphenol; 6-(4-hydroxyphenoxy)-2,4-bis(n-ocytlthio)-1,3,5-triazine; 2,4,6-tris(4-hydroxy-3,5-di-tert-butyl-phenoxy)-1,3,5- -triazine; di-n-octadecyl-3,5-di-tert-butyl-4-ydroxybenzylphosphonate; 2-(n-octylthio)ethyl-3,5-di-tert-butyl-4-hydroxybenzoate; and sorbitol hexa-(3,3,5-di-tert-butyl-4-hydroxy-phenyl)propionate. Proprietary commercial stabilizers and/or antioxidants are available under a number of trade names including a variety of Wingstay®, Tinuvin® and Irganox® products.

Various bacteriostats are known in the art and may be included as additives within the slow recovery elastomer. Examples of suitable bacteriostats include benzoates, phenols, aldehydes, halogen containing compounds, nitrogen compounds, and metal-containing compounds such as mercurials, zinc compounds and tin compounds. A representative bacteriostat is 2,4,4′-trichloro-2′-hydroxy-diphenyl-ether which is available under the trade designation Irgasan Pa. from Ciba Specialty Chemical Corporation, Tarrytown, N.Y.

Other optional additives include thermoplastic polymers or thermoplastic polymer compositions which preferentially associate with the hard blocks or segments of the block copolymers. Without intending to be bound by theory, it is believed that these thermoplastic polymers become incorporated into the entangled three-dimensional network structure of the hard phase. This entangled network structure can provide improved tensile, elastic and stress relaxation properties of the elastomeric composition. Where the elastomeric polymer comprises a styrenic block copolymer, thermoplastic polymer additives such as polyphenylene oxide and vinylarene polymers derived from monomers including styrene, alpha-methyl styrene, para-methyl styrene, other alkyl styrene derivatives, vinyl toluene, and mixtures thereof, are useful in the present invention because they are generally considered to be chemically compatible with the styrenic hard blocks of the block copolymer.

Various viscosity modifiers, processing aids, slip agents or anti-block agents can be employed as additives to yield a slow recovery elastomer with, for example, improved handling characteristics or surface characteristics. Processing aids include processing oils, which are well known in the art and include synthetic and natural oils, naphthenic oils, paraffinic oils, olefin oligomers and low molecular weight polymers, vegetable oils, animal oils, and derivatives of such including hydrogenated versions. Processing oils also may incorporate combinations of such oils. A particularly preferred processing oil is mineral oil. Viscosity modifiers are also well known in the art. For example, petroleum derived waxes can be used to reduce the viscosity of the slow recovery elastomer in thermal processing. Suitable waxes include low number-average molecular weight (e.g., 600-6000) polyethylene; petroleum waxes such as paraffin wax and microcrystalline wax; atactic polypropylene; synthetic waxes made by polymerizing carbon monoxide and hydrogen such as Fischer-Tropsch wax; and polyolefin waxes.

Various colorants and fillers are known in the art and may be included as additives within the slow recovery elastomer. Colorants can include dyes and pigments such as titanium dioxide. Fillers may include such materials as talc and clay. Other additives may include dyes, UV absorbers, odor control agents, perfumes, fillers, dessicants, and the like.

The slow recovery elastomers of the present invention exhibit unique elastic and recovery characteristics. The slow recovery elastomer exhibits a normalized unload force of greater than about 0.04 N/mm² at 37C as measured by the Two Cycle Hysteresis Test. Normalized unload forces of less than about 0.04 N/mm² at 37C. are believed to be insufficient for use as an elastomer within absorbent articles. Materials having normalized unload forces less than 0.04 N/mm² at 37C are unable to keep an absorbent article in snug, close contact to the wearer's skin. Preferably, the slow recovery elastomer exhibits a normalized unload force of greater than about 0.08 N/mm² at 37C, and, most preferably, exhibits a normalized unload force of greater than about 0.12 N/mm² at 37C.

Traditional elastomers (i.e., those commonly used in disposable absorbent articles such as Vector 4211 from Dexco Polymers L. P., Houston, Tex.) exhibit minimal post elongation strain at 22C after 15 seconds of recovery. Qualitatively, traditional elastomers exhibit “snap back” (i.e., the elastomer contracts relatively quickly after being released from a stretched state). In the case of a diaper comprising an elasticized topsheet with a conventional elastomer, upon stretching and release of the diaper, the elastomer contracts relatively quickly, causing the diaper to fold, thus making it difficult to position and apply the diaper successfully. In contrast, the slow recovery elastomers of the current invention exhibit at least about 20% post elongation strain at 22C after 15 seconds of recovery, as measured by the Post Elongation Recovery Test. In other embodiments, the slow recovery elastomer exhibits at least about 50% post elongation strain after 15 seconds of recovery at 22C In other suitable embodiments, at 22C the slow recovery elastomer exhibits a post elongation strain from about 75% to about 150% after 15 seconds of recovery. However, post elongation strain after 15 seconds of recovery may exceed about 170% at 22C.

Furthermore, the slow recovery elastomers of the present invention may exhibit a specified post elongation strain at 22C after 30 seconds, 60 seconds, or three minutes of recovery. In certain embodiments, the slow recovery elastomer may exhibit at least about a 70% post elongation strain after 30 seconds of recovery at 22C. In other embodiments, the slow recovery elastomer may exhibit at least about a 40% post elongation strain after 60 seconds of recovery at 22C.

The slow recovery elastomer may exhibit temperature responsiveness. In one embodiment, a temperature responsive slow recovery elastomer may exhibit a post elongation strain after 15 seconds at 32C. that is at least 35% less than the post elongation strain after 15 seconds at 22C. Preferably, at least a 50% reduction in post elongation strain is exhibited. Most preferably, at least a 75% reduction in post elongation strain is exhibited. It is believed that a slow recovery elastomer exhibiting temperature responsiveness may further facilitate diaper application. When the diaper is applied at about room temperature (e.g., approximately 22C), the slow recovery elastomer exhibits a relatively high degree of post elongation strain for a prescribed period of time. Upon application of the diaper, the slow recovery elastomer will rise in temperature because of the close proximity of the wearer's skin. As the temperature of the slow recovery elastomer increases and nears body temperature (e.g., approximately 32C), the reduced post elongation strain is exhibited. Temperature responsiveness allows for application of the diaper without “snap-back” while providing for increased recovery after application.

The slow recovery elastomer of the present invention may exist in a variety of forms. The slow recovery elastomer forms include, but are not limited to films, bands, strands, individualized fibers, or combinations thereof. Furthermore, the slow recovery elastomer may take any of the previous forms and be further combined with a traditional elastic not exhibiting the unique rate of recovery of the present invention (i.e., an elastic not exhibiting at least about 50% post elongation strain after 15 seconds of recovery at 22C). The slow recovery elastomer may be utilized in a variety of articles. However, the composition has particular benefit within absorbent articles, particularly in the handles 200 of the absorbent article 20, as described in greater detail above. The slow recovery elastomer may also be used in place of or in addition to traditional elastomers commonly present in the absorbent article 20. The slow recovery elastomer may be used discretely or be may be joined to another material or substrate (such as a polymeric film, a nonwoven, a woven, or a scrim).

### Test Methods

(1) Post Elongation Recovery

This method is used to determine the post elongation strain of an elastomer as a function of temperature and time. The measurement is done at 22C (72° F.) or at 32C (90° F.). The measurement at 22C (72° F.) is designed to simulate the recovery of the elastomer at room temperature, while the measurement at 32C (90° F.) is designed to measure the recovery of the elastomer near skin temperature. A two-step analysis, Stretch and Recovery, is performed on the samples. The method employs a Dynamic Mechanical Analyzer (DMA) such as a TA Instruments DMA 2980 (hereinafter “DMA 2980”), available from TA Instruments, Inc., of New Castle, Del.; equipped with a film clamp, Thermal Advantage/Thermal Solutions software for data acquisition, and Universal Analysis 2000 software for data analysis. Many other types of DMA devices exist, and the use of dynamic mechanical analysis is well known to those skilled in the art of polymer and copolymer characterization.

Methods of operation, calibration and guidelines for using the DMA 2980 are found in TA Instruments DMA 2980 Operator's Manual issued March 2002, Thermal Advantage User's Reference Guide issued July 2000 and Universal Analysis 2000 guide issued February 2003. To those skilled in the use of the DMA 2980, the following operational run conditions should be sufficient to replicate the stretch and recovery of the samples.

The experimental conditions are selected on the DMA 2980 which specify operation in the Controlled Force Mode with the film clamp. The film clamp is mounted onto the DMA 2980 and calibrated according to the User's Reference Guide. The material to be tested is cut into samples of substantially uniform dimension. Appropriate sample dimensions may be selected to achieve the required strain. For the DMA 2980, suitable sample dimensions are approximately 6.4 mm wide by approximately 0.15 mm thick. The floating film clamp of the DMA 2980 is adjusted to a position which provides approximately 6 mm between the clamping surfaces, and is locked in this position. The sample is mounted in the film clamps and the lower clamp is allowed to float to allow determination of the actual gauge length which exists between the film clamps. The sample ID and dimensions are recorded. The furnace is closed.

Stretch Method—Specific DMA 2980 parameter settings for the above sample dimensions are set as follows: Preload force applied to sample in clamp (0.01N); auto zero displacement (on) at the start of the test; furnace (close), clamp position (lock), and temperature held at Ti (22C or 32C) at the end of the stretch method. Data acquisition rate is set at 0.5 Hz (1 point per 2 seconds). The stretch method is loaded onto the DMA 2980. The method segments are (1) Initial Temperature Tᵢ (22C or 32C), (2) Equilibrate at Tᵢ (3) Data Storage ON, and (4) Ramp Force 5.0 N/min to 18.0 N.

Upon initiation of the test, the temperature ramps to the specified Tᵢ (22C or 32C) [method segment 1], and the temperature is maintained at this Tᵢ [method segment 2]. After a minimum of 15 minutes at Tᵢ, the operator initiates the sample stretching and concurrent data collection [method segments 3 and 4]. The sample is stretched with an applied ramp force of 5 N per minute to approximately 30 mm in length. The gradual increase in force more closely simulates application of the article and prevents breakage. The sample is locked in place at the stretched length of approximately 30 mm and maintained at Tᵢ. The force required to reach the 400% strain is recorded manually from the digital readout on the instrument.

For samples of different dimensions, the applied force is adjusted to achieve an applied ramp force of 5 N/min per square millimeter of initial sample cross-sectional area; and the maximum displacement is adjusted to achieve a strain of 400%. The percent strain is calculated by subtracting the gauge length from the stretched length, then dividing the result by the gauge length and multiplying by 100. A sample stretched from an initial length of 6 mm to a length of 30 mm results in a 400% strain.

Recovery Method—The Recovery Method is loaded onto the instrument and initiated 15 seconds after reaching the desired strain (400%) in the Stretch Method. The four segments of the recovery method are (1) Data Storage ON, (2) Force 0.01N, (3) Ramp to Tᵢ, and (4) Isotherm for 3.0 minutes. The following DMA 2980 parameter setting is changed from the Stretch Method: auto zero displacement is changed to (OFF). The Recovery Method measures the length of the sample over a 3 minute time period at the specified temperature (Tᵢ=either 22C or 32C). The sample length, percent strain, and test temperature are recorded as a function of recovery time. The post elongation strain is reported as percent strain after different times of recovery (15 seconds, 30 seconds, 60 seconds, and 3 minutes).

For different sample dimensions, the force is adjusted to achieve 0.01 N/per square millimeter of initial sample cross-sectional area (determined prior to stretching the sample).

(2) Two Cycle Hysteresis Test

This method is used to determine properties that may correlate with the forces experienced by the consumer during application of the product containing the elastomeric composition and how the product fits and performs once it is applied.

The two cycle hysteresis test method is performed at room temperature (21C/70° F.) and also at body temperature (37C/99° F.). The material to be tested is cut into a substantially rectilinear shape. Sample dimensions should be selected to achieve the required strain with forces appropriate for the instrument. Suitable instruments for this test include tensile testers commercially available from MTS Systems Corp., Eden Prairie, Minn. (e.g., Alliance RT/1 or Sintech 1/S) or from Instron Engineering Corp., Canton, Mass. For either the Alliance RT/1 or Sintech 1/S instruments listed above, suitable sample dimensions are approximately 0.13 mm thick, approximately 20 mm wide by approximately 100 mm long.

The following procedure illustrates the measurement when using the above sample dimensions and either an Alliance RT/1 or Sintech 1/S. The instrument is interfaced with a computer. TestWorks 4™ software controls the testing parameters, performs data acquisition and calculation, and provides graphs and data reports.

The grips used for the test are wider than the sample. Typically 1″ (2.54 cm) wide grips are used. The grips are air actuated grips designed to concentrate the entire gripping force along a single line perpendicular to the direction of testing stress having one flat surface and an opposing face from which protrudes a half round (radius=6 mm) to minimize slippage of the sample. In the case of the measurement at 37C, the upper grip is a lightweight grip with serrated faces.

The load cell is selected so that the forces measured will be between 10% and 90% of the capacity of the load cell or the load range used. Typically a 25 N load cell is used. The fixtures and grips are installed. The instrument is calibrated according to the manufacturer's instructions. The distance between the lines of gripping force (gauge length) is 2.50″ (63.5 mm), which is measured with a steel ruler held beside the grips. The load reading on the instrument is zeroed to account for the mass of the fixture and grips. The mass and thickness of the specimen are measured before testing. The specimen is mounted into the grips in a manner such that there is no slack and the load measured is between 0.00N and 0.02N. After being mounted in the grips, the sample is equilibrated at the testing temperature for 5 minutes before starting the test. A suitable environmental chamber is used to maintain the temp at 37C for measurements performed at this temperature. The instrument is located in a temperature-controlled room for measurements performed at 21C.

The two cycle hysteresis test method involves the following steps:

(1) Strain the sample to the specified maximum percent strain (i.e., Strainₘₐₓ=150%) at a constant crosshead speed of 20″/min. (50.8 cm/min) with no hold.

(2) Reduce strain to 0% strain (i.e., return grips to original gauge length of 2.50″) at a constant crosshead speed of 3″/min. (7.62 cm/min) with no hold.

(3) Strain the sample to Strainₘₐₓ at a constant crosshead speed of 20″/min. (50.8 cm/min) with no hold.

(4) Reduce strain to 60% strain at a constant crosshead speed of 3″/min. (7.62 cm/min)

(5) Hold at 60% strain for 5 minutes.

(6) Go to 0% strain at a constant crosshead speed 3″/min. (7.62 cm/min)

The measured unload force is the force at 60% strain after the 5 minute hold in step 5.

This force is normalized to Newtons per square millimeter of initial sample cross-sectional area (determined before the sample is stretched) as follows: Normalized unload force=measured unload force/[(initial sample thickness in mm)×(initial sample width in mm)]

For different sample dimensions, the crosshead speed is adjusted to maintain the appropriate strain rate for each portion of the test. For example; a crosshead speed of 10″/min (25.4 cm/min) would be used in Steps 1 and 3 for a sample gauge length of 1.25″ (31.7 mm).

### EXAMPLES

Exemplary films are prepared by blending varying amounts of elastomeric polymer, modifying resin and mineral oil as shown in Table 1. The blending is accomplished by extrusion of the mixture (Examples 2 and 3) or by solvent casting the mixture and pressing into a film on a heated Carver Press (Examples 1, 4, 5, 6 and 7). The amount of each component is expressed in weight percent of the elastomeric composition. The examples in Table 1 comprise a triblock elastomeric copolymer, styrene-isoprene-styrene (S-I-S), commercially available under the trade designation Vector 4211 from Dexco Polymers L.P., Houston, Tex. In some examples (Examples 2, 3, 4, 6 and 7), a component of the elastomeric composition is white mineral oil, commercially available under the trade designation Britol® 50T from Crompton Corporation, Petrolia, Pa. Modifying resins suitable for use that are disclosed in the examples in Table 1 are an alicyclic hydrocarbon resin under the trade designation Arkon P140 (Tg of 86C), available from Arakawa Chemical Inc., Chicago, Ill., and poly(t-butyl styrene) (Tg of 126C and 130C for the 14 kDa and 19 kDa resins, respectively), synthesized at Procter & Gamble Company via free radical polymerization of t-butylstyrene monomer available from Aldrich Chemical Company. The weight average molecular weights of the poly(t-butylstyrene) samples are 14 and 19 kDa as determined by gel permeation chromatography using polystyrene standards in tetrahydrofuran.

TABLE 1
Elastomeric Composition (Weight %)

| | Sample | | | | | | |
|---|---|---|---|---|---|---|---|
| | 1* | 2 | 3 | 4 | 5 | 6 | 7 |
| Vector 4211 (SIS, 29% S) | 100 | 45 | 45 | 49 | 60 | 59 | 45 |
| Poly(t-butyl styrene), 14 kDa | | 45 | | | | | |
| Poly(t-butyl styrene), 19 kDa | | | 45 | | 40 | 39 | 45 |
| Arkon P140 | | | | 49 | | | |
| Mineral Oil, White Britol-50T | | 10 | 10 | 2 | | 2 | 10 |

*Sample 1 is a comparative example whereas Sample 2-7 are embodiments of the present invention.

Films of the elastomeric compositions in Table 1 are measured according to the Post Elongation Recovery method described in the Test Methods section above. The thickness of the film tested and the force (not normalized for film thickness) in Newtons to strain the sample to 400% strain are shown in Table 2. The post elongation strain is reported at different recovery times (15 seconds, 30 seconds, 60 seconds, and 3 minutes).

TABLE 2
Post Elongation Strains of Films of the Elastomeric Compositions of Table 1

| | Sample | | | | | | |
|---|---|---|---|---|---|---|---|
| | 1 | 2 | 3 | 4 | 5 | 6 | 7 |
| Elongation Strain at 22 C. (72° F.) | | | | | | | |
| Film Thickness (mm) | 0.16 | 0.13 | 0.14 | 0.14 | 0.16 | 0.16 | 0.13 |
| % Strain after 15 seconds recovery | 13 | 137 | 178 | 102 | 153 | 104 | 157 |
| % Strain after 30 seconds recovery | 13 | 98 | 141 | 73 | 117 | 70 | 122 |
| % Strain after 60 seconds recovery | 12 | 64 | 105 | 50 | 88 | 44 | 88 |
| % Strain after 3 minutes recovery 11 | 11 | 29 | 59 | 28 | 48 | 19 | 48 |
| Force (N) | 2.5 | 1.0 | 1.3 | 1.1 | 1.7 | 1.1 | 1.2 |
| Post Elongation Strain at 32 C. (90° F.) | | | | | | | |
| Film Thickness (mm) | 0.15 | 0.14 | 0.13 | 0.14 | 0.16 | 0.14 | 0.14 |
| % Strain after 15 seconds recovery | 16 | 43 | 109 | 41 | 58 | 56 | 81 |
| % Strain after 30 seconds recovery | 15 | 24 | 74 | 17 | 37 | 34 | 53 |
| % Strain after 60 seconds recovery | 15 | 13 | 46 | 18 | 23 | 21 | 33 |
| % Strain after 3 minutes recovery 11 | 14 | 7 | 19 | 11 | 11 | 11 | 16 |
| Force (N) | 1.8 | 0.6 | 0.7 | 0.7 | 1.0 | 0.7 | 0.8 |

The normalized unload forces of films of the elastomeric compositions in Table 1 are measured at 21C and 37C according to the 2-Cycle Hysteresis Test described in the Test Methods Section above. The data are shown in Table 3.

TABLE 3
Normalized Unload Forces (N/mm²) of Films of
Elastomeric Compositions of Table 1

| | Sample | | | | | | | |
|---|---|---|---|---|---|---|---|---|
| | 1 | 2 | 3 | 4 | 5 | 6 | 7 | 8¹ |
| Normalized | 0.50 | 0.06 | 0.08 | 0.13 | 0.15 | 0.17 | 0.10 | N.A. |
| Unload Force | | | | | | | | |
| at 21 C. | | | | | | | | |
| (70° F.) | | | | | | | | |
| [N/mm²] | | | | | | | | |
| Normalized | 0.63 | 0.08 | 0.08 | 0.12 | 0.14 | 0.16 | 0.10 | 0.03 |
| Unload Force | | | | | | | | |
| at 37 C. | | | | | | | | |
| (99° F.) | | | | | | | | |
| [N/mm²] | | | | | | | | |

¹Sample No. 8 is a comparative example of Findley H2401 adhesive (pressed into a film using a heated Carver Press).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a chassis comprising:
a backsheet,
a topsheet joined to the backsheet, and
an absorbent core disposed between the backsheet and the topsheet; and
at least one handle joined to the chassis, the handle comprising a slow recovery elastomer wherein the slow recovery elastomer exhibits a normalized unload force of greater than about 0.04 N/mm² at 37C after a 5 minute hold time at 60% strain and a post-elongation strain of at least about 30% after 15 seconds of recovery at 22C.

2. The absorbent article according to claim 1, wherein the slow recovery elastomer exhibits a post-elongation strain of at least about 80% after 15 second of recovery at 22C.

3. The absorbent article according to claim 1, wherein the chassis has a waist region, and the at least one handle is disposed in the waist region.

4. The absorbent article according to claim 3, wherein the chassis includes ears, and the at least one handle is disposed proximate to the ears.

5. The absorbent article according to claim 4, wherein the chassis includes a pair of front ears and a pair of rear ears, one each of the front ears fastened to one each of the rear ears to define a pair of joined front and rear ears, and the at least one handle disposed across one of the pairs of joined front and rear ears.

6. The absorbent article according to claim 4, wherein includes a pair of front ears and a pair of rear ears, one each of the front ears fastened to one each of the rear ears to define a pair of joined front and rear ears, and the at least one handle disposed to one side or the other of the pair of joined front and rear ears.

7. The absorbent article according to claim 1, wherein the article comprises another handle in addition to the at least one handle.

8. The absorbent article according to claim 1, wherein the at least one handle comprises at least one flap.

9. The absorbent article according to claim 1, wherein the at least one handle comprises at least one tab.

10. The absorbent article according to claim 1, wherein the at least one handle comprises at least one ridge.

11. The absorbent article according to claim 1, wherein the at least one handle comprises at least one loop.

12. The absorbent article according to claim 1, wherein the at least one handle comprises at least one region.

13. An absorbent article comprising:
a chassis comprising:
a backsheet,
a topsheet joined to the backsheet, and
an absorbent core disposed between the backsheet and the topsheet; and
at least one handle joined to the chassis, the handle comprising a material that is capable of transmitting at least 30-50% of the force applied and that recovers to substantially or nearly substantially its initial shape after forces are applied to the handle are removed.

14. The absorbent article according to claim 13, wherein the material is capable of transmitting at least about 20N to the chassis.

15. A method of using an absorbent article, the absorbent article having a chassis comprising a backsheet, a topsheet joined to the backsheet, and an absorbent core disposed between the backsheet and the topsheet, the chassis fastened to define leg openings and a waist opening; and at least one handle joined to the chassis and shaped to facilitate grasping by one of limited dexterity, the method comprising:
disposing a wearer's legs through the waist opening and the leg openings;
grasping the at least one handle;
applying a force to the chassis via the at least one handle to move the article upward along the legs toward the wearer's waist, at least 30-50% of the force applied to the at least one handle being transmitted to the chassis;
releasing the at least one handle, the at least one handle subsequently recovering to substantially or nearly substantially its initial shape.

16. The method according to claim 15, wherein the handle is capable of transmitting at least about 20N.

17. The method according to claim 15, wherein the handle is made of a slow recovery elastomer, the slow recovery elastomer exhibiting a normalized unload force of greater than about 0.04 N/mm² at 37C after a 5 minute hold time at 60% strain and a post-elongation strain of at least about 30% after 15 seconds of recovery at 22C.

18. The absorbent article according to claim 17, wherein the slow recovery elastomer exhibits a post-elongation strain of at least about 80% after 15 second of recovery at 22C.
